(19) United States
(12) Patent Application Publication
(10) Pub. No.: US 2025/0064717 A1
(43) Pub. Date: Feb. 27, 2025

(54) MANUFACTURE OF COSMETIC CAROTENOID COMPOSITIONS

(71) Applicant: Unibar Corporation, Houston, TX (US)

(72) Inventor: Sevanti Mehta, Houston, TX (US)

(21) Appl. No.: 18/727,758

(22) PCT Filed: Jan. 10, 2023
(86) PCT No.: PCT/US23/10486

§ 371 Date: Jul. 10, 2024

Related U.S. Application Data
(60) Provisional application No. 63/298,804, filed on Jan. 12, 2022.

(51) Int. Cl. A61K 8/9789; A61K 36/81; A61Q 5/00; A61Q 19/00

(52) U.S. Cl. CPC A61K 8/9789; A61K 36/81; A61Q 5/006; A61Q 19/00; A61K 2236/37; A61K 2800/92

(57) ABSTRACT

A capsicum annum extract composition (600) includes capsanthin in the range from 1%-1.5%, zeaxanthin in the range from 0.5% to 1%, and cryptoxanthin in the range from 0.1% to 0.5%. The composition is created, at least in part, from material spent from purifying carotenoids using counter current extractions, and the composition enhances skin, eyes, or hair.

### CROSS-REFERENCE TO RELATED APPLICATIONS

This application claims the benefit of U.S. Provisional Application No. 63/298,804, filed Jan. 12, 2022 and titled “Manufacture of Cosmetic Carotenoid Compositions” by Sevanti Mehta.

### SUMMARY

A capsicum annum extract composition includes capsanthin in the range from 1%-1.5%, zeaxanthin in the range from 0.5% to 1%, and cryptoxanthin in the range from 0.1% to 0.5%. The composition is created, at least in part, from material spent from purifying carotenoids using counter current extractions, and the composition enhances skin, eyes, or hair.

A method for manufacturing a composition includes extracting carotenoids from spent material, the spent material created from purifying carotenoids from capsicum annum fruits using counter current extractions using a solvent or solvents. The method further includes enriching the extracted carotenoids using super critical fluid extraction. The method further includes purifying the enriched carotenoids using counter current extractions such that the composition enhances skin, eyes, or hair.

A method for manufacturing a composition includes extracting carotenoids from spent material, the spent material created from purifying carotenoids from capsicum annum fruits using counter current extractions using a solvent or solvents. The method further includes enriching the extracted carotenoids using super critical fluid extraction. The method further includes purifying the hydrolyzed carotenoids using counter current extractions such that the composition includes capsanthin in the range from 1% to 1.5%, zeaxanthin in the range from 0.5% to 15%, and cryptoxanthin in the range from 0.1% to 0.5% such that the composition enhances skin, eyes, or hair.

### BRIEF DESCRIPTION OF THE DRAWINGS

Accordingly, compositions and methods are provided according to one or more of the following examples. In the drawings:

FIG. 1 is an illustrative method of manufacturing a composition;

FIG. 2 is an illustrative bar chart showing lowering of intraocular pressure;

FIGS. 3-5 are illustrative diagrams depicting the results of a study to evaluate anti-obesity effect;

FIG. 6 is an illustrative composition;

FIG. 7 is an illustrative bar chart showing melanin inhibition;

FIG. 8 is an illustrative bar chart showing tyrosinase inhibition; and

FIG. 9 is an illustrative bar chart showing gene expression analysis of gene transcripts for fold increase.

It should be understood, however, that the specific embodiments given in the drawings and detailed description thereto do not limit the disclosure. On the contrary, they provide the foundation for one of ordinary skill to discern the alternative forms, equivalents, and modifications that are encompassed together with one or more of the given embodiments in the scope of the appended claims.

### DETAILED DESCRIPTION

Certain terms are used throughout the following description and claims to refer to particular system components and configurations. As one of ordinary skill will appreciate, companies may refer to a component by different names. This document does not intend to distinguish between components that differ in name but not function. In the following discussion and in the claims, the terms “including” and “comprising” are used in an open-ended fashion, and thus should be interpreted to mean “including, but not limited to . . . ”.

Carotenoids are any of various pigments such as carotenes found widely in plants and animals and characterized chemically by a long aliphatic polyene chain composed of eight isoprene units. Capsanthin is a carotenoid, as are beta-carotene, lycopene, lutein, and zeaxanthin. These pigments are found in red, orange, yellow, and green fruits and vegetables. Carotenoids are antioxidant tetraterpenoids, which aid disease-prevention mechanisms of the body.

Capsicums are a widely consumed natural foodstuff used as a vegetable, spice, and/or color, and paprika extract is an extract of the fruits of the genus capsicum. The genus, which originates from Central and Southern America, belongs to the solanaceae family and includes all peppers, from the mild bell pepper to the spicy habanero. There are five domesticated species of capsicum: annum, frutescens, chinense, pubescens, and baccatum, and the most widely spread are annum, frutescens, and chinense. The first to be introduced worldwide was annum, originating from Mexico. It was previously divided into two categories: sweet (or mild) peppers and hot (or chili) peppers, though modern plant breeding removed that distinction. At present, annum is the most wide-spread in terms of household consumption and industrial processing.

Annum is the varietal used to manufacture paprika extract for food coloration. Color extracts have a low content of capsaicin compared with the extracts used as spice agents. In the red varietal, capsanthin and capsorubin are the main compounds responsible for the red color. Pure carotenoid crystals derived from the annum fruits include xanthophylls such as capsanthin, zeaxanthin, and cryptoxanthin. The chemical structure of the carotenoid ultimately determines what potential biological function(s) that pigment may have. The distinctive pattern of alternating single and double bonds in the polyene backbone of carotenoids is what allows them to absorb excess energy from other molecules, while the nature of the specific end groups on carotenoids may influence their polarity. The carbonyl group present in capsanthin and capsorubin makes them unique when compared to other carotenoids such as lutein and zeaxanthin.

Age-related macular degeneration (“AMD”) is a common medical eye condition and a leading cause of vision loss among people over the age of fifty. It includes damage to the macula, an oval-shaped pigmented area near the center of the retina that is used for sharp, central vision. AMD may be of two types: dry (atrophic) or wet (exudative). In addition to aging, high blood-pressure, oxidative stress, blue light exposure, obesity, and the like may also cause AMD. The carotenoids in the macula help filter blue wavelengths of sunlight and reduce free radicals near the retina both of which are harmful to eye cells. Lutein and zeaxanthin are the carotenoids present in the retina, and they protect eye cells from photo oxidative damage. Specifically, chronic exposure to blue light, the major cause of AMD, causes reduction in cone density and cone sensitivity, and zeaxanthin may prevent AMD by absorbing blue light.

Skincare is not only a cosmetic challenge but also a major health concern especially considering the aging population with significant exposure to sunlight. Skincare products are therapeutic formulations intended for contact with the various external parts of the human body and protect against degenerative dermal disorders. Synthetic chemicals used in a variety of cosmetic formulations are associated with undesirable and sometimes irreversible side effects, and also cause skin allergy. The petroleum-based chemicals, such as parabens and phthalates, are recognized as health hazards, and due to this, consumers demand natural herbal-based ingredients. Additionally, synthetic fragrances are linked to reproductive toxicity, cancer, and endocrine disruption. Accordingly, most prefer cosmetics derived from natural products over synthetic chemicals, as natural products are devoid of undesirable side effects. Cosmetic dermatology application of capsanthin is disclosed via in vitro experiments such as sun protection factor (“SPF”), melanin inhibition activity, tyrosinase inhibition activity, effectiveness against microorganism-caused acne and dandruff, and inducing the gene expression of Aquaporin 3 (“AQP-3”) in the human keratinocyte cell line.

SPF is a laboratory measure of the efficacy of protecting against ultraviolet (“UV”) radiations. Exposure to UV radiation causes sunburn and hyperpigmentation. The in vitro SPF is determined by the spectrophotometric method, and the higher the SPF, the more the protection against UV radiation. Melanin pigments are produced and transferred by melanocytes to the surrounding keratinocytes. The special cells, melanocytes, are located at the base layer of the epidermis. Melanin is primarily responsible for skin pigmentation, and has a vital role in the prevention of UV radiation-induced skin damage. The enzymes tyrosinase, tyrosinase-related protein 1 (“TRP1”), and dopachrome tautomerase (“DCT”), are responsible for the enzymatic conversion of tyrosine to melanin. The synthesis of melanin is accelerated by various factors, including UV radiation and cAMP elevating phytochemical forskolin from Coleus forskohlii. The abnormal biosynthesis of melanin can result in significant aesthetic issues such as melasma, freckles, hyperpigmentation, and age spots. Accordingly, the disclosed compositions and methods display skin brightening properties by inhibition of melanin biosynthesis. Specifically, the depigmentation is achieved by diminishing the proliferation of melanocytes or the inhibition of biosynthesis of melanin by inhibiting tyrosinase, a copper-containing enzyme.

The lipophilic Malassezia furfur belongs to a monophyletic genus of fungi that is typically found in human and animal skin. More than 80% of the human skin fungal population constitutes Malassezia furfur, and is frequently isolated in both infected and healthy hosts. Malassezia spp. has been associated with several common dermatologic disorders, including pityriasis versicolor, seborrheic dermatitis, and Malassezia folliculitis. Malassezia may contribute to other dermatological conditions such as atopic dermatitis and psoriasis. Skin condition can be improved by reducing the number of Malassezia with antifungal agents. For example, dandruff is a skin condition characterized by a flaky, itchy scalp, and accompanied by colonization of the skin by Malassezia spp. The most common and well-studied disease-causing Candida spp. is Candida albicans, which naturally colonizes the skin, intestinal mucosa, and genitals in healthy humans. High morbidity and mortality rates have been reported due to severe infection, especially in hospitalized patients.

As disclosed herein, two types of capsanthin extracts obtained from red bell pepper fruit, also known as red paprika, offer dermal protection against all the conditions described above. The capsanthin extracts are characterized by high-performance liquid chromatography (“HPLC”). Specifically, capsanthin obtained from red bell pepper fruits is effective in UV protection, melanin inhibition, skin brightening, tyrosinase inhibition, and inducing gene expression of Aquaporin 3 (AQP-3) in the human keratinocyte cell line (HaCaT). Capsanthin 50% w/w crystals (“CAP-50CR”) and capsanthin 1.5% w/w soft extract (“CAP-1.5SE”) both show these effects.

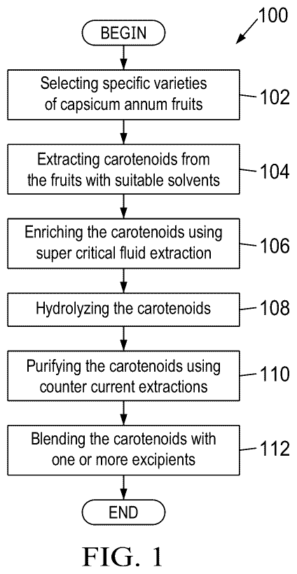

FIG. 1 illustrates a method 100 of manufacturing the two substances. Specifically, carotenoid compositions in accordance with some illustrated embodiments with the benefits described above. Also disclosed herein are methods for isolating and purifying carotenoids containing a specific composition of carotenoids such as trans-capsanthin, trans-zeaxanthin, and beta-cryptoxanthin from capsicum annum fruits leaving no trace of organic hazardous solvents. The method 100 includes selection of high colored composition of annum chili varieties, solvent extraction, super critical fluid extraction (“SCFE”) enrichment, alkali hydrolysis of carotenoid esters with absolute alcohol, purification using counter current extractor, concentration, and drying. The resulting crystals are compositions suitable for cosmetics, dietary supplements, food additives, and the like.

At 102, specific varieties of capsicum annum fruits are selected. In an embodiment, the capsicum annum fruit is selected from the Bydagi chili varieties, or other chilies high in color value, alone or in combination such as: Bydagi-Kaddi, Bydagi-Dyavnoor, Bydagi-Dabbi, KDL high color chili, 5531 high color chili, and/or 4431 high color chili. In an embodiment, the ratio of the combination is: (1) Bydagi-Kaddi: (1) Bydagi-Dyavnoor: (1) Bydagi-Dabbi (a ratio of 1:1:1). In other embodiments, unexpected results have been found using the ratio of 1:2:2 and 1:1:2 of Bydagi-Kaddi: Bydagi-Dyavnoor: Bydagi-Dabbi. Additionally, unexpected results have been found using the ratio of KDL high color, 5531 high color, and 4431 high color in 1:1:2, respectively. In various embodiments, the American Spice Trade Association (“ASTA”) color value of the Bydagi chilies, or other high color chilies, are selected within the range from 2000 to 2600 units or a range from 2000 to 2400 units.

At 104, carotenoids are extracted from the fruits with suitable solvents. In an embodiment, dried, deseeded, and flaked capsicum annum fruits undergo extraction using a solvent at a temperature ranging from 40° C. to 90° C., preferably at 60° C., for 4 hours to 8 hours, preferably 6 hours. The solvent extract is then concentrated under vacuum to produce capsicum oleoresin containing carotenoids esters. In various embodiments, the extraction solvent may be one or a combination of: methanol, ethanol, and/or isopropyl alcohol.

For example, 250 kilograms of deseeded, flaked capsicum annum fruits with ASTA color value from 2000 to 2400 units may be placed in a 2000 liter capacity reactor with an agitator. A volume of methanol (1000L) may be added and the mixture may be stirred for 6 hours at 60° C. The methanol layer may be filtered and collected. This methanol extraction may be repeated three times for efficiency purposes. As another example, dried red bell pepper flakes (Capsicum annum) may be extracted with n-hexane at 40° C. to 60° C. for 6 hours, filtered, and concentrated under a vacuum.

At 106, the carotenoids are enriched using super critical fluid extraction. In an embodiment, carbon dioxide is used as a solvent, and the temperature for super critical fluid extraction ranges from 40° C. to 60° C., preferably 50° C. In an embodiment, the pressure employed for super critical fluid extraction ranges from 25 to 50 mPa, preferably 35 mPa.

Continuing the above example, all the methanol layers may be combined and concentrated under vacuum. 22 kg of oleoresin may be obtained and formulated with approximately 110 kg of calcium carbonate in super critical fluid extractor bags. The extractor bags may be placed in an extraction chamber and extracted at 50° C. and 35 mPa. The yield of enriched oleoresin may be approximately 11 kg.

At 108, the carotenoid esters are hydrolyzed. In an embodiment, the enriched oleoresin is hydrolyzed with alcoholic potassium hydroxide to produce free carotenoids. In an embodiment, the base for hydroxylation is selected from a group consisting of potassium hydroxide (KOH), sodium hydroxide (NaOH), or a combination thereof. In an embodiment, the solvent media for hydrolysis is methanol, ethanol, isopropyl alcohol, or a combination thereof. In an embodiment, concentration of the hydrolysis agent ranges from 10% to 30%. In an embodiment, the temperature of hydrolysis ranges from 70° to 85° C., preferably 80° C. In an embodiment, the time required for hydrolysis ranges from 1 hour to 3 hours, preferably 2 hours. As the carotenoids are hydrolyzed to free form, they become more bioavailable.

Continuing the above example, the enriched oleoresin may be placed in a 500 L glass lined reactor. In a separate vessel, 5 kg of Potassium hydroxide may be added to 40 L of ethyl alcohol while stirred. The alcoholic KOH may be added to the enriched oleoresin slowly while stirred at 80° C. for two hours.

At 110, the hydrolyzed carotenoids are purified using counter current extractions. In an embodiment, the solvent used for counter current extraction is ethyl acetate, isopropyl acetate, or a combination thereof. In an embodiment, the immiscible aqueous phase is water or water acidified with hydrochloric acid (pH 3-4), preferably acidified water.

Continuing the above example, after ensuring the degree of saponification to be more than 99% by HPLC, 40 liters of demineralized hot water maintained at a temperature of 70° C. may be added to the reacted mass while stirred for 10 minutes. The diluted mass with carotenoid crystals may be pumped into a filter press to recover the crystals. Around 250 liters of additional hot water may be pumped through the filter press to wash the unwanted impurities and bring down the pH of the effluent to neutral around 7.0. After ensuring the neutralization, a positive pressure of nitrogen may be applied to the filter press to squeeze the crystals trapped inside the filter. The wet crystals with an approximate weight 6.7 kg may then be collected from the filter press, dissolved in 60 L of ethyl acetate, and charged into a counter current extractor. 100 L of water may be used, and the pH may be adjusted to 3 or 4 with dil-HCl. The acidified water may be bottom fed into the counter current extractor.

As another example, the obtained oleoresin may be further purified by supercritical fluid extraction and saponified using 40% ethanolic caustic potash. The saponified extract may then be concentrated to remove ethanol and further purified by ethyl acetate/water mixture.

At 112, the capsicum annum extract is blended with excipient(s). In various embodiments, the excipient is sunflower oil, safflower oil, soy lecithin, sunflower lecithin, phosphatidylcholine from sunflower or soy, starch, dextrin, lactose, dicalcium phosphate, colloidal silicon dioxide, and/or combinations thereof. In other embodiments, the excipient is a granulating agent, binding agent, lubricating agent, disintegrating agent, sweetening agent, glidant, anti-adherent, anti-static agent, surfactant, anti-oxidant, gum, coating agent, coloring agent, flavoring agent, coating agent, plasticizer, preservative, suspending agent, emulsifying agent, plant cellulosic material, spheronization agent, and/or combinations thereof. In an embodiment, the carotenoids are further isolated and/or purified by: addition of one or more solvents, addition of ionic resin, quenching, filtration, extraction, and/or ion exchange resin.

Continuing the example above, the ethyl acetate layer may be removed, dried over anhydrous sodium sulphate, and concentrated. The yield of composition may be about 1.1 kg. The carotenoid contents as measured by a spectrophotometer may be about 95.34%, with all trans-capsanthin, all trans-zeaxanthin, and all the beta-cryptoxanthin by HPLC at 98.71%, 8.31% and 2.63% respectively. The final product may contain a moisture content of about 0.2% with no traces of residual methanol and ethyl acetate detected by gas chromatography analysis.

As another example, the ethyl acetate layer may be separated, dried over anhydrous sodium sulfate, and concentrated under vacuum to obtain capsanthin 50% crystals (“CAP-50CR”). The aqueous layer, usually industrial waste, may contain a small amount of capsanthin. The pH of the aqueous layer may be adjusted to 5.5 to 7.0 with dilute hydrochloric acid, and ethyl acetate may be added and heated to 55 to 60° C. The separated ethyl acetate layer may be removed, dried over anhydrous sodium sulfate, and concentrated to obtain capsanthin 1.5% soft extract (“CAP-1.5SE”).

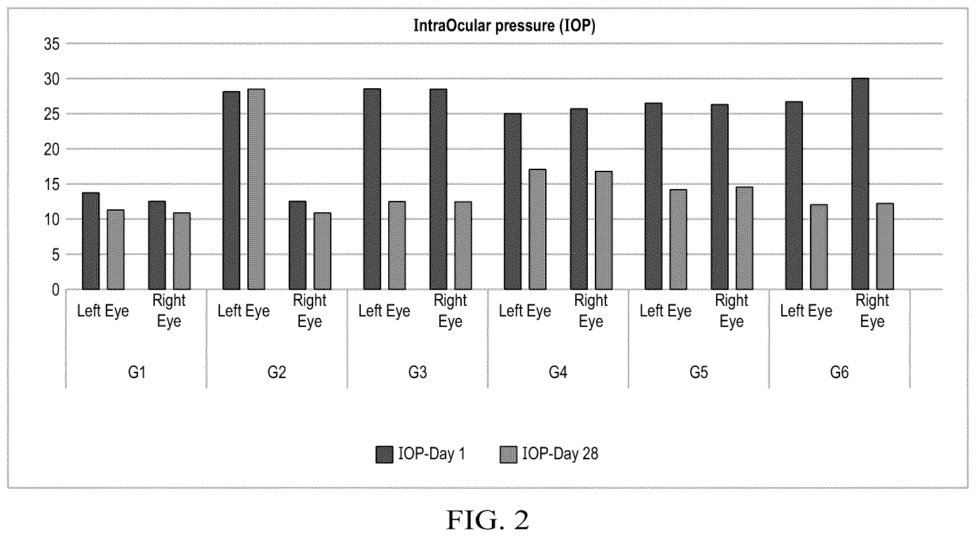

FIG. 2 depicts the results of a study using the disclosed compositions for lowering of intraocular pressure (“IOP”) in animals, and any of the steps below may be part of the method 100 in various embodiments. The high intraocular pressure originated from an increased resistance to drainage of aqueous humor through the trabecular meshwork. A sustained increase in aqueous humor may be due to an increase in the formation of aqueous humor, a difficulty in its exits, or a raised pressure in the episcleral vein. In this study, IOP was induced by intravitreal injection. FIG. 2 shows a bar chart depicting six groups (G1-G6), and the left eye and right eye IOP for each group. The first two groups (G1-G2) are control groups in which no IOP lowering composition was introduced. In G1, IOP was not induced, and in G2, IOP was induced. For the next for groups (G3-G6), the left bar for each eye represents IOP prior to introduction of the composition disclosed herein in one embodiment. The right bar for each eye represents IOP after introduction of the composition. The results show that introduction of the compositions reduced IOP to a level almost equal to the group in which IOP was not induced (G1), and significantly lower than the group in which the composition was not introduced (G2).

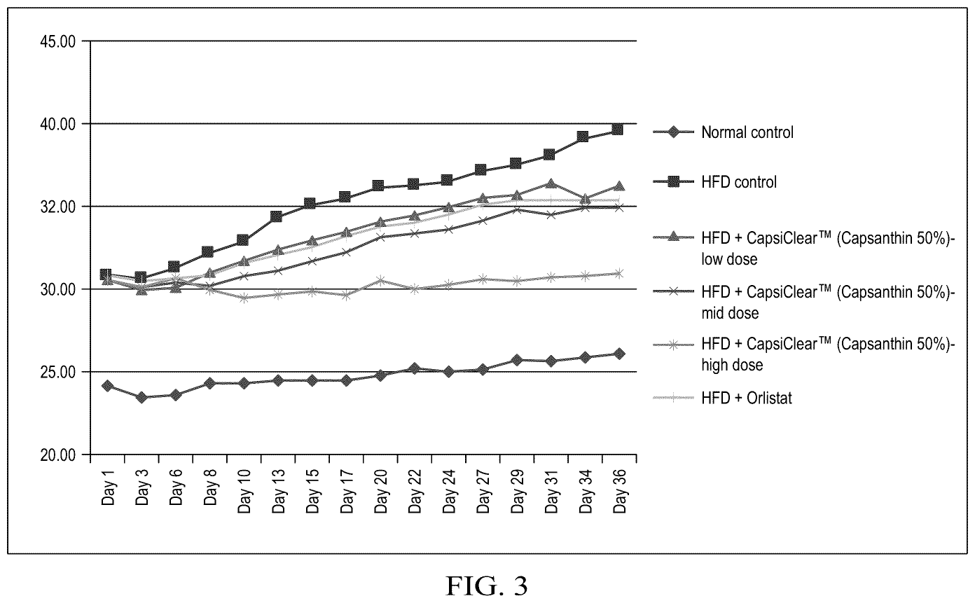

FIGS. 3 to 5 depict the results of a study to evaluate anti-obesity effect of the compositions disclosed herein, referred to in the Figures as CapsiClear, and any of the steps below may be part of the method 100 in various embodiments. “HFD” refers to a high-fat diet used to induce obesity. Six groups were used in this study: G1-G6. G1 refers to the group given a normal diet, G2 refers to the group given a high-fat diet, G3 refers to the group given a high-fat diet with a low dose of the composition, G4 refers to the group given a high-fat diet with a medium dose of the composition, G5 refers to the group given a high-fat diet with a high dose of the composition, and G6 refers to the group given a high-fat diet with a reference drug Orlistat.

In FIG. 3, the y-axis depicts body weight while the x-axis depicts time in days. As can be seen, a significant reduction in body weight of 31% was observed at the high dose of 80 mg/kg.

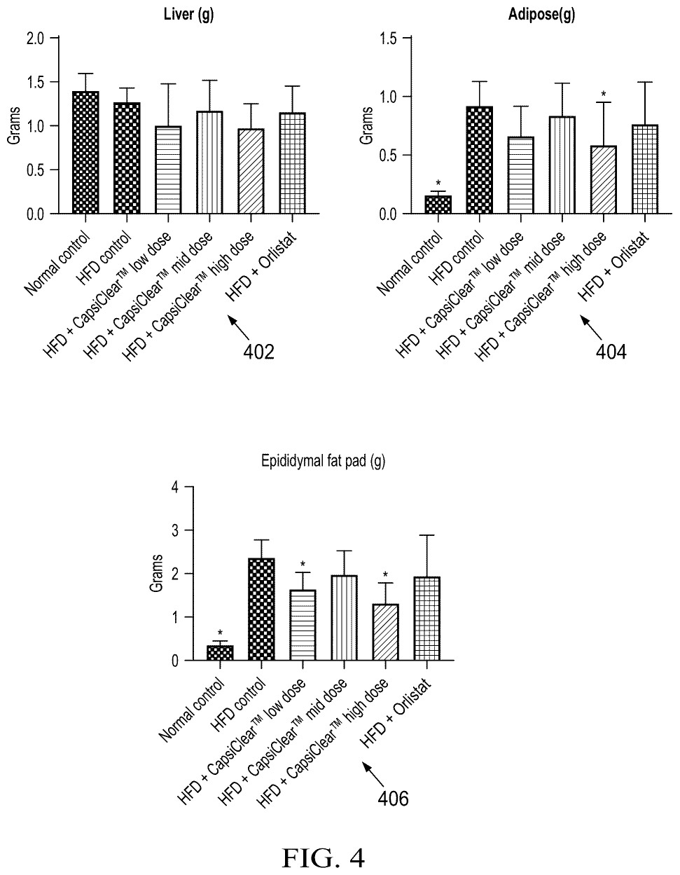

FIG. 4 includes three charts 402, 404, 406 showing the results of different measurements in the study, and any of the steps below may be part of the method 100 in various embodiments. At 402, liver mass was decreased in groups given the composition. At 404, adipose tissue weight was significantly reduced in the group given high doses of the composition. At 406, epididymal fat pad weight was significantly reduced in the group low and high doses of the composition.

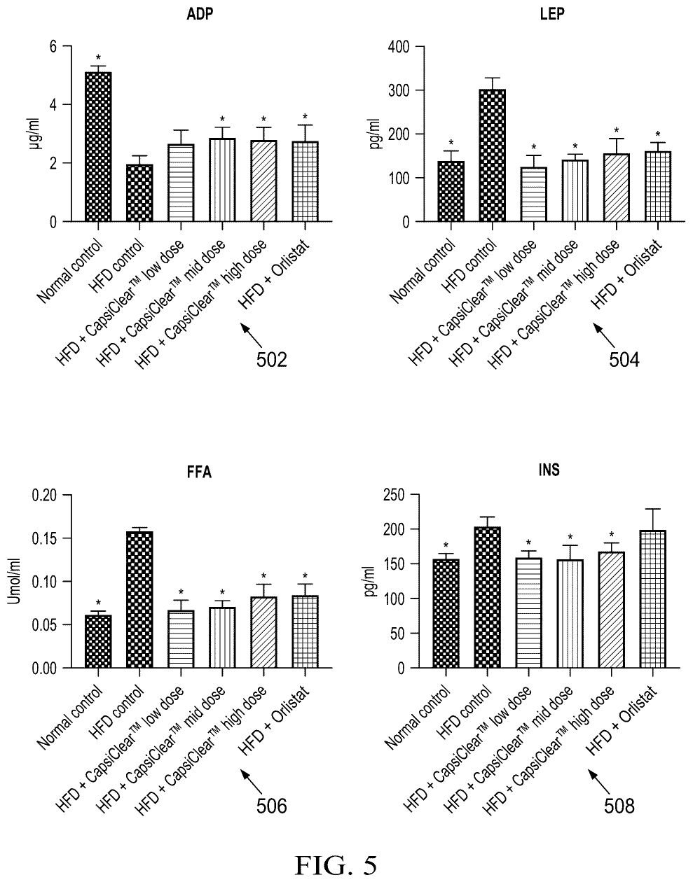

FIG. 5 includes four charts 502, 504, 506, 508 showing the results of different measurements in the study, and any of the steps below may be part of the method 100 in various embodiments. At 502, adiponectin was significantly increased in the groups given medium and high doses of the composition. The increase in adiponectin causes a decrease in insulin-resistance and body weight. At 504, 506, and 508, plasma leptin, plasma free fatty acid, and insulin respectively were significantly reduced in all groups given the composition. The lowering of insulin causes the lowering of body weight, the lowering of plasma free fatty acid causes improved mitochondrial function and whole-body insulin sensitivity, and the lowering of plasma leptin causes restoration of hypothalamic leptin sensitivity, reduction in weight gain, and enhancement of insulin sensitivity. Accordingly, based on all of the above, the compositions may be used effectively for weight loss purposes as well along or in combination with other benefits.

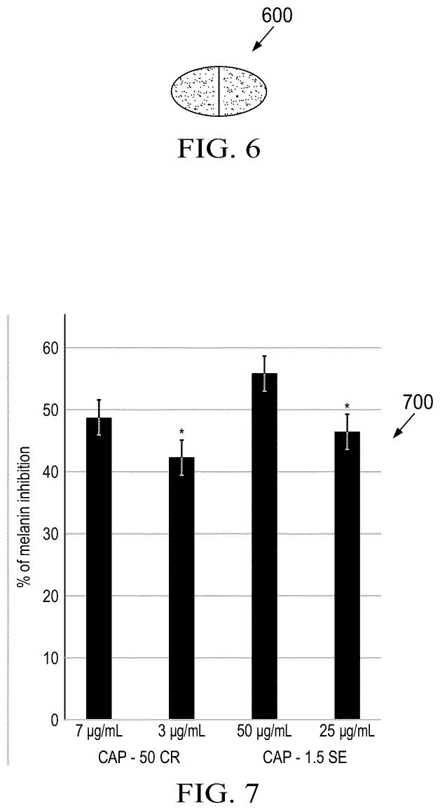

FIG. 6 illustrates a composition 600 of the capsicum annum extract including capsanthin, zeaxanthin, and cryptoxanthin manufactured as described above in a capsule embodiment for pharmaceutical, nutraceutical, and/or cosmeceutical purposes. In other embodiments the composition is implemented as a tablet, injectable, cream, gel, ointment, lotion, solution, beverage, confection, emulsion, foam, troche, lozenge, aqueous suspension, oily suspension, patch, dentifrice, spray, drop, powder, granule, syrup, elixir, food stuff, and/or combinations thereof. In preferred embodiments, the composition includes a tablet, soft gelatin capsule, hard gelatin capsule, cream, gel, lotion, and/or combinations thereof.

In an embodiment, the composition may be administered by topical administration, oral administration, intravenous administration, intra articular administration, intramuscular administration, and/or combinations thereof in various embodiments. In preferred embodiments, the mode of administration is oral, topical, intravenous intramuscular, and/or combinations thereof. In addition, materials such as flaxseed, flaxseed oil, vegetarian or vegetable oil, and other oils may be combined with the composition to stabilize the active ingredients. Regardless of the amount of materials combined with the composition, the percentages of carotenoids in relation to the total amount of carotenoids remains the same in at least some embodiments.

A 12-week, randomized, placebo-controlled, pilot clinical study indicates that the composition may: increase macular pigment density (“MPOD”), which is linked to better visual performance, reduce recovery time after exposure to bright light, and improve reading under different light conditions (both white and blue light) in addition to the benefits already described.

The compositions may include capsanthin in the range from 1%-1.5%, zeaxanthin in the range from 0.5% to 1%, and cryptoxanthin in the range from 0.1% to 0.5%. The compositions may be created, at least in part, from material spent from purifying carotenoids using counter current extractions, and the compositions enhance skin, eyes, or hair. As a result of the above process, in an embodiment the compositions of carotenoids ranges from capsanthin: 50% to 80%, zeaxanthin: 5% to 15%, cryptoxanthin: 1% to 5%, and trace amounts of other carotenoids. These percentages, and all percentages herein, refer both to actual percentages and percentages allowable by labeling regulations. In an embodiment, the compositions of total carotenoids ranges from 90% to 99%. In another embodiment, the compositions may include total carotenoids in the range from 10% to 99%. In an embodiment, the carotenoids are trans-capsanthin: (3R,3′S,5′R)-3,3′-dihydroxy-β,κ-caroten-6′-one; trans-zeaxanthin: 3R,3′R-β,β-carotene-3,3′-diol; and beta-cryptoxanthin: (3R,6′R)-4′,5′-didehydro-5′,6′-dihydro-β,β-caroten-3-ol. The capsanthin may include trans-capsanthin (3R,3′S,5′R)-3,3′-Dihydroxy-β,κ-caroten-6′-one), the zeaxanthin may include trans-zeaxanthin (3R,3′R-β,β-carotene-3,3′-diol), and the cryptoxanthin may include beta-cryptoxanthin (3R,6′R)-4′,5′-Didehydro-5′,6′-dihydro-β,β-caroten-3-ol.

In an embodiment, the color value of carotenoids ranges from 800,000 to 1,250,000. Accordingly, the composition is ideal to provide color and skin enhancing features to cosmetics such as lipstick, chap-stick, liquid gloss, lipstick paste, blush, lip liner, foundation, concealer, eye contourer, eyeliner, mascara, nail polish, eye shadow, body make-up, and the like. WHO FAO, JECFA, and HPLC analysis may be performed to identify and quantify capsanthin in CAP-50CR and CAP-1.5SE using a reference standard. In the HPLC chromatogram of the retention time of capsanthin in reference standard, CAP-50CR and CAP-1.5SE were identical, which confirms the presence of capsanthin. In various embodiments, the method 100 includes any of the steps below.

Presently, a steady increase in solar ultraviolet radiation is implicated in non-melanoma skin cancer reported worldwide. The UV radiation present in solar light has been implicated as its main cause. UV radiation is known to cause excess reactive oxygen species (“ROS”), thereby increasing oxidative stress, which induces skin damage and non-melanoma skin cancer. Thus, reducing ROS and oxidative stress is key to preventing skin from skin damage and cancer.

Carotenoids, the major class of polyene compounds in biology, and long polyene bonds present in the molecules scavenge free radicals by quenching the excited molecules. Capsanthin is a highly potent carotenoid due to the presence of a keto group in conjunction with eleven conjugated dienes in its structure. The conjugated dienes are responsible for their radical scavenging capacity and singlet oxygen quenching capability. Skin protective, anti-cancer, anti-inflammatory, anti-obesity, anti-cholesterol, and anti-adipogenicity activities exhibited by capsanthin are a consequence of its potent radical quenching ability.

The effectiveness of a sun protection agent is determined by the photometric sun protection factor (“SPF”). Effective sunscreen agents must have a wide range of absorbance (290 and 400 nm to protect the skin from UV radiation and skin damage. As per the US Food and Drug Administration, a sun protection agent should have a minimum of 15 SPF. The SPF value for CAP-50CR and CAP-1.5SE, the compositions disclosed herein, are 34.44 and 20.63, respectively. These values qualify CAP-50CR and CAP-1.5SE as high and medium sun protection agents, respectively. The following table shows the SPF trials and averages. For CAP-50CR, the photometric SPF value measured 34.58, 33.62, and 35.13 in three trials for an average of 34.44. For CAP-1.5SE, the photometric SPF value measured 21.96, 19.96, and 19.97 over three trials for an average of 20.63.

Photometric SPF
Value

| Test Item | Trial 1 | Trial 2 | Trial 3 | Average |
|---|---|---|---|---|
| CAP-50CR | 34.58 | 33.62 | 35.13 | 34.44 |
| CAP-1.5SE | 21.96 | 19.96 | 19.97 | 20.63 |

In addition to SPF, the composition also exhibits antifungal properties as shown in the table below. The anti-fungal activity of CAP-50CR and CAP-1.5SE against Malassezia furfur and Candida albicans are compared with the anti-fungal drug ketoconazole by the minimum inhibitory concentration (“MIC”) method.

The inhibitory effect assay of CAP-50CR and CAP-1.5SE against Malassezia furfur and Candida albicans may be performed as per a culture microdilution reference method. Ketoconazole, an anti-fungal drug, may be used as the standard. Freeze-dried Candida albicans (ATCC-10231) and Malassezia furfur (ATCC-14521) strains may be used as well. A loopful culture of Malassezia furfur and Candida albicans may be streaked on SDA (Sabouraud Dextrose Agar), and incubated at 35±2° C. and 25±2° C. for 48-72 hours, respectively. Finally, cells may be suspended in a RPMI 1640 medium to obtain a final concentration of 1×106 CFU/mL. For the evaluation of inhibitory activity, the effect of CAP-50CR and CAP-1.5SE may be examined against strains of Malassezia furfur and Candida albicans cultured in 96-well microplates at different concentrations of 35±2° C. and 25±2° C., respectively, for 48-72 hours.

The antifungal standard ketoconazole showed an MIC value of 0.00048 mg/mL against both Malassezia furfur and Candida albicans. The MIC for CAP-50CR and CAP-1.5SE against Malassezia furfur was found to be 0.625 mg/mL and 5 mg/mL, respectively. CAP-50CR and CAP-1.5SE showed the same MIC value against Candida albicans, which is 2.5 mg/mL. Notably, CAP-50CR extract displayed a moderate antifungal activity, and CAP-1.5SE showed a mild anti-fungal activity compared to the antifungal drug ketoconazole. Accordingly, the composition may aid in hair growth and the composition may aid in removing and preventing dandruff.

| | | MIC Range | MIC₅₀ |
|---|---|---|---|
| Species | Test Item | (mg/mL) | (mg/mL) |
| Malassezia furfur | Ketoconazole | 1 to 0.00048 | 0.00048 |
| | CAP-50CR | 10 to 0.0048 | 0.625 |
| | CAP-1.5SE | 10 to 0.0048 | 5 |
| Candida albicans | Ketoconazole | 1 to 0.00048 | 0.00048 |
| | CAP-50CR | 10 to 0.0048 | 2.5 |
| | CAP-1.5SE | 10 to 0.0048 | 2.5 |

Sterility and growth control may also be used. Yeast growth may be determined in an Enzyme-Linked ImmunoSorbent Assay Microplate Reader at 530 nm. Minimum inhibitory concentration (MIC50) may be defined as the lowest concentration of CAP-50CR and CAP-1.5SE that produces a reduction of 50% of the fungal growth compared to controls. Accordingly, the composition may additionally be an anti-yeast agent against Malassezia yeast.

The compositions may aid in melanin inhibition as shown in FIG. 7. Mouse skin melanoma cell lines B16F10 may be cultured in a DMEM-HG medium supplemented with 10% inactivated fetal bovine serum (“FBS”), penicillin (100 IU/mL), streptomycin (100 μg/mL), and amphotericin B (5 μg/mL), in a humidified atmosphere of 5% CO₂ at 37° C. until confluent. The cells may be dissociated with TPVG solution (0.2% trypsin, 0.02% EDTA, 0.05% glucose in PBS). The stock cultures may be grown in 25 cm² culture flasks, and 96-well microtiter plates may be used.

The monolayer cell culture may be trypsinized, and the cell count may be adjusted to 1.0×105 cells/mL using a DMEM-HG medium containing 10% FBS. 0.1 mL of the diluted cell suspension added to each well and kept for 24 hours. After the monolayer is formed, the supernatant may be flicked off, washed with medium, and 100 μl of test substances with different concentrations may be added and incubated at 37° C. for 48 hours in a 5% CO2 atmosphere. A microscopic examination may be carried out, and observations may be noted at every 24 hour time interval.

Cell viability may be assessed by MTT reduction assay. After 72 hours of incubation, the drug solutions may be discarded, and 50 μl of MTT in PBS may be added to each well. The plates may be gently shaken and incubated for 3 hours at 37° C. in a 5% CO2 atmosphere. The supernatant may be removed and 100 μl of DMSO may be added. The plate may be gently shaken to solubilize the formed formazan. The absorbance may be measured using a microplate reader at a wavelength of 540 nm. The concentration of test substances needed to inhibit the growth of the cell by 50% (CTC50) may be calculated.

The cytotoxicity of CAP-50CR and CAP-1.5SE in B16F10 cell lines may be assessed to determine CTC50 values. The 24 hour cell culture, which may be 70-80% confluent, may be used to determine melanin inhibition. The cells may be treated with non-toxic concentrations of the test substance and forskolin (200 μM) for 48 hours.

At the end of the incubation period, the supernatants may be aspirated from wells,

and cultures may be washed twice with warm PBS. The cells may be harvested by trypsinization, and the cell suspension may be centrifuged at 2500 rpm for 15 minutes. The cell pellets may be washed with PBS, centrifuged, and treated with 400 μL of IN NaOH containing 10% DMSO. Samples may be heated at 60° C. for 1 hour, cooled, and the absorbance of the cell lysates may be estimated spectrophotometrically at 450 nm.

The in vitro cytotoxicity of CAP-50CR and CAP-1.5SE may be evaluated on skin melanoma cells (B16F10) by MTT assay. Different concentrations ranging from 1000 μg/mL to 7.8 μg/mL may be used to determine the percentage growth inhibition on B16F10 cells. CAP-50CR shows a CTC50 value of 98.44±2.55 μg/mL on the B16F10 cell line, whereas CAP-1.5SE exhibits a CTC50 value greater than 1000 μg/mL. Furthermore, the non-toxic concentrations of the items may be evaluated for forskolin-induced melanin inhibition activity. The cytotoxic properties of the substances are given in Table 3, and the percentage of melanin inhibition is given in FIG. 7. For CAP-50CR, the non-toxic concentrations of 7 and 3 μg/mL shows melanin inhibition of 48.73±0.75% and 42.29±5.2%, respectively. The melanin inhibition for CAP-1.5SE is 55.84%±1.47 and 46.44%±1.05 for the non-toxic concentrations of 50 and 25 μg/mL, respectively.

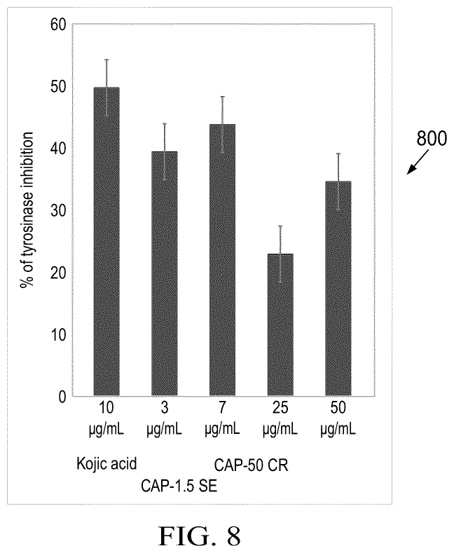

The compositions may inhibit tyrosinase as shown in FIG. 8. CAP-50CR and CAP-1.5SE exhibited high tyrosinase inhibitory activity at non-toxic concentrations. As such, they are a depigmentation agent for cosmetic and drug applications. Specifically, the excessive production of melanin leads to pigmentation disorders due to abnormal melanogenesis, and tyrosinase is a significant marker for developing curative agents of pigment disorder and skin brightening.

Tyrosinase inhibitory activity may be measured by using DOPA oxidase activity of mushroom tyrosinase determined spectrophotometrically. 10 μL of DMSO may be taken in a 96-well microplate and mixed with 60 μL of 50 mmol/L phosphate buffer (pH 6.8) on ice. Next, 20 μL of 0.9 mg/mL L-DOPA in phosphate buffer may be added. Finally, 10 μL of tyrosinase (500 U/mL in phosphate buffer) may be added to the mixture and incubated at 27° C. for 10 minutes. After incubation, the amount of dopachrome production in the reaction mixture may be determined spectrophotometrically at 450 nm (OD450) in a microplate reader. Kojic acid (5-100 μmol/L) dissolved in 50 mmol/L phosphate buffer may be used as a positive control. The concentration for 50% inhibition (IC50) may be determined.

The CAP-50CR extract exhibits a greater tyrosinase inhibition activity than that of CAP-1.5SE. The CAP-50CR extract shows 43.78% and 39.37% inhibition at the concentrations of 7 and 3 μg/mL, respectively. CAP-1.5SE shows 34.6% and 22.9% for the concentrations of 50 and 25 μg/mL, respectively. Accordingly CAP-50CR and CAP-1.5SE demonstrate skin brightening effect by inhibiting tyrosinase.

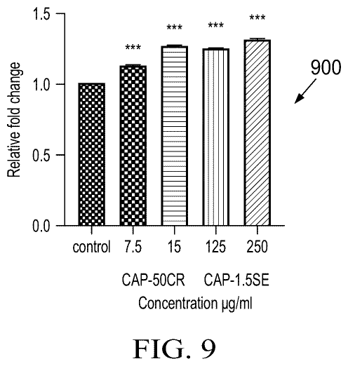

The compositions may induce gene expression in human keratinocyte (HaCaT) as shown in FIG. 9. Keratinocytes comprising the epidermis produce loricrin, involucrin, and filaggrin to combine keratin filaments and form a cornified cell envelope. In addition, hyaluronic acid present in keratinocytes acts as the moisturizing barrier in the skin. The water retention rate of skin and other organs is regulated by Aquaporins (AQPs), small aquaphobic protein molecules. As of today, in mammals, almost 13 types of AQPs have been identified. The function of AQP3 is considered as important for the skin as it involves the transport of glycerol and water. In response to skin damage, AQP is downregulated. As such, AQP plays an important role in restoring skin functions, and is involved in the biosynthesis of hyaluronic acid (HA). A decrease in HA in the skin leads to wrinkles, loss of elasticity, and premature aging. The effect of CAP-50CR and CAP-1.5SE on the expression of gene encoding AQP3 in HaCaT cells may be examined using RT-PCR.

Stock cells may be cultured in DMEM supplemented with 10% inactivated fetal bovine serum (FBS), penicillin (100 IU/mL), streptomycin (100 μg/mL), and amphotericin B (5 μg/mL) in a humidified atmosphere of 5% CO2 at 37° C. until confluent. The cells may be dissociated with TPVG solution (0.2% trypsin, 0.02% EDTA, 0.05% glucose in PBS).

The monolayer cell culture may be trypsinized, and the cell count may be adjusted to 100,000 cells/mL using 10% FBS, and 0.1mL of diluted cell suspension may be taken in a 96-well microtiter plate. After the formation of the monolayer, the supernatant may be flicked off, washed with medium, and 100 μl test substances with different concentrations may be added and incubated at 37° C. for 72 hours in a 5% CO2 atmosphere. After 72 hours, the test solutions may be discarded, and 50 μl of MTT in PBS may be added to each well. The plates may be gently shaken and incubated for 3 hours at 37° C. in a 5% CO2 atmosphere. The supernatant may be removed, and 100 μl of DMSO may be added to dissolve the formazan. The absorbance may be measured using a microplate reader at a wavelength of 540 nm. The percentage growth inhibition and concentration of test substance needed to inhibit cell growth by 50% (CTC50) may be calculated.

HaCaT cells may be subjected to cell lysis by treating them with TriExtract reagent. Chloroform may be added and centrifuged to isolate the total RNA from the sample. The upper layers may be collected, diluted with an equal volume of isopropanol, and incubated at −20° C. for 10 minutes. After the incubation, the centrifuged and appropriate volume of ethanol may be added to resuspend. The isolated RNA pellets may be air-dried, and TAE buffer may be added. The first-strand cDNA may be synthesized from RNA using oligo dT primers followed by reverse transcriptase enzyme treatment. The cDNA may be taken up for PCR for the amplification of AQP-3 and GAPDH.

The mRNA expression levels of AQP-3 may be determined by using a semi-quantitative reverse transcriptase polymerase chain reaction (RT-PCR) procedure. The AQP-3 may be amplified with the following primers. For I strand synthesis: Oligo dT primer. For II strand synthesis: forward 5′-GCTGTCACTCTGGCATCCTG-3′ and reverse: 5′-GCGTCTGTGCCAGGGTGTAG-3′. The amplification reactions may be performed with the following scheme: 5 minutes at 95° C. followed by 35 cycles of denaturation at 95° C. for 30 seconds, annealing temperature for 30 seconds, and 72° C. for 45 seconds with a 10 minute final extension at 72° C. for 10 minutes.

CAP-50CR and CAP-1.5SE, exhibit CTC50 values of 76.59±3.25 μg/mL and 664.95±2.88 μg/mL, respectively, on the HaCaT cells. Furthermore, in non-toxic concentrations CAP-50CR and CAP-1.5SE show an increase in the levels of AQP-3 mRNAs at lower and higher concentrations as compared to a control in a semi-quantitative RT-PCR procedure. FIG. 9 is a graph of semi-quantitative gene expression analysis of gene transcripts for fold increase, the relative level of AQP-3 gene expression is normalized to GAPDH. Values are expressed as mean±SEM (n=2), and statistical significance is compared between control versus other treatment groups (CAP-50CR 7.5 μg/mL CAP-50CR 15 μg/mL, CAP-1.5SE 125 μg/mL and CAP-1.5SE 250 μg/mL) *** p<0.0001, statistically significant. Both substances showed an increase in the levels of AQP-3 mRNAs at lower and higher concentrations as compared to the control in the semi-quantitative RT-PCR procedure.

Based on the above, the compositions may aid in skin moisturization, restoring skin function by increasing the biosynthesis of hyaluronic acid. The compositions may increase levels of AQP-3 mRNAs. The compositions may aid in protecting skin from ultraviolet radiation, UVA radiation, and UVB radiation. The compositions may provide a sun protection factor (SPF) of at least 20. The compositions may prevent and treat dry eye syndrome when administered orally. The compositions may increase tear break up time and reduce tear osmolarity when administered orally. The compositions may increase tear production volume when administered orally. The compositions may decrease defects and abrasions in the corneal epithelium when administered orally. The compositions may reduce damage to ocular surfaces by reducing oxidative stress when administered orally. The compositions may reduce degradation and apoptotic cell death in the cornea by reducing serum MMP-2& MMP-9 levels when administered orally. The compositions may restore the mucin layer of the eye when administered orally. The compositions may reduce the expression of the IL-2, IL-6, and MMP-9 genes. The compositions may down regulate the expression of the TNF-α and IL-4 genes.

CAP-50CR and CAP-1.5SE exhibit skin protection from UV radiation and hyper-pigmentation properties. They also exhibit anti-fungal, skin brightening, anti-wrinkle, and moisturizing properties. These results show that capsanthin from red bell pepper fruits can be employed as a cosmetically active ingredient in skin guard formulations and as a potential therapeutic agent for a variety of dermatological disorders.

Additional embodiments and features of the present disclosure will be apparent to one of ordinary skill in art based upon description provided herein. The embodiments herein and the various features and advantageous details thereof are explained with reference to the non-limiting embodiments in the description. The examples used herein are intended merely to facilitate an understanding of ways in which the embodiments herein may be practiced and to further enable those of skill in the art to practice the embodiments herein. Accordingly, the following examples should not be construed as limiting the scope of the embodiments herein.

In some aspects, compositions and methods are provided according to one or more of the following examples:

Example 1: A capsicum annum extract composition includes capsanthin in the range from 1%-1.5%, zeaxanthin in the range from 0.5% to 1%, and cryptoxanthin in the range from 0.1% to 0.5%. The composition is created, at least in part, from material spent from purifying carotenoids using counter current extractions, and the composition enhances skin, eyes, or hair.

Example 2: A method for manufacturing a composition includes extracting carotenoids from spent material, the spent material created from purifying carotenoids from capsicum annum fruits using counter current extractions using a solvent or solvents. The method further includes enriching the extracted carotenoids using super critical fluid extraction. The method further includes purifying the enriched carotenoids using counter current extractions such that the composition enhances skin, eyes, or hair.

Example 3: A method for manufacturing a composition includes extracting carotenoids from spent material, the spent material created from purifying carotenoids from capsicum annum fruits using counter current extractions using a solvent or solvents. The method further includes enriching the extracted carotenoids using super critical fluid extraction. The method further includes purifying the hydrolyzed carotenoids using counter current extractions such that the composition includes capsanthin in the range from 1% to 1.5%, zeaxanthin in the range from 0.5% to 15%, and cryptoxanthin in the range from 0.1% to 0.5% such that the composition enhances skin, eyes, or hair.

The following features may be incorporated into the various embodiments described above, such features incorporated either individually in or conjunction with one or more of the other features:

The capsanthin may include trans-capsanthin (3R,3′S,5′R)-3,3′-dihydroxy-β,κ-caroten-6′-one), the zeaxanthin may include trans-zeaxanthin (3R, 3′R-β,β-carotene-3,3′-diol), and the cryptoxanthin may include beta-cryptoxanthin (3R,6′R)-4′,5′-Didehydro-5′,6′-dihydro-β,β-caroten-3-ol. The color value of carotenoids may range from 800,000 to 1,250,000. The composition may aid in the management of age related macular degeneration conditions such as blurred vision, distorted vision, reduced central vision, and difficulty in adopting low light levels. The composition may protect against blue light induced retinopathy via decreasing oxidative and endoplasmic reticulum stress. The composition may provide color for a cosmetic such as lipstick, chap-stick, liquid gloss, lipstick paste, blush, lip liner, foundation, concealer, eye contourer, eyeliner, mascara, nail polish, eye shadow, or body make-up. The composition may provide functional and morphological preservation of photoreceptors against blue light damage. The composition may lower the intraocular pressure associated with tension glaucoma, primary open angle glaucoma, and angle closure glaucoma. The composition may lower body weight. The composition may lower insulin thereby lowering body weight. The composition may lower plasma free fatty acid thereby improving mitochondrial function and whole-body insulin sensitivity. The composition may lower plasma leptin thereby restoring hypothalamic leptin sensitivity, reducing weight gain, and enhancing insulin sensitivity. The composition may increase adiponectin thereby decreasing insulin-resistance and body weight. The composition may decrease adipose tissue weight and epididymal fat pad. The composition may be in a form such as a capsule, tablet, injectable, cream, gel, ointment, lotion, solution, beverage, confectionery, emulsion, foam, troche, lozenge, aqueous suspension, oily suspension, patch, dentifrice, spray, drop, powder, granule, syrup, elixir, or food stuff. The composition may include total carotenoids in the range from 10% to 99%. The capsicum annum fruits may be the KDL high color, 5531 high color, and 4431 high color in a 1:1:2 ratio, respectively. The ASTA color value of the capsicum annum fruits may range from 2000 to 2600 units. The solvents may include ethanol, methanol, and isopropyl alcohol. Extracting the carotenoids may include extracting the carotenoids at a temperature ranging from 40° C. to 90° C. for a time period ranging from 4 hours to 8 hours. The solvent media for super critical fluid extraction may be carbon dioxide. Enriching the carotenoids may include enriching the carotenoids using super critical fluid extraction at a temperature ranging from 40° C. to 60° C. Enriching the carotenoids may include enriching the carotenoids using super critical fluid extraction at a pressure ranging from 25 mPa to 50 mPa. Hydrolyzing the carotenoids may include hydrolyzing the carotenoids using KOH and NaOH. Hydrolyzing the carotenoids may include hydrolyzing the carotenoids using Methanol, Ethanol, and Isopropyl alcohol. Hydrolyzing the carotenoids may include hydrolyzing the carotenoids using a hydrolysis agent within the range of 10% to 30%. Hydrolyzing the carotenoids may include hydrolyzing the carotenoids at a temperature within the range of 70° C. to 85° C. Hydrolyzing the carotenoids may include hydrolyzing the carotenoids within the range of 1 hour to 3 hours. Purifying the carotenoids may include purifying the carotenoids using a solvent for counter current extraction comprising ethyl acetate and isopropyl acetate. An immiscible aqueous phase may be used for counter current extraction water and water acidified with hydrochloric acid (pH 3-4). The methods may further include blending the carotenoids with an excipient such as sunflower oil, safflower oil, soy lecithin, sunflower lecithin, phosphatidylcholine from sunflower or soy, starch, dextrin, lactose, dicalcium phosphate, or colloidal silicon dioxide. The capsanthin may include trans-capsanthin (3R,3′S,5′R)-3,3′-Dihydroxy-β,κ-caroten-6′-one), the zeaxanthin may include trans-zeaxanthin (3R, 3′R-β,β-carotene-3,3′-diol), and the cryptoxanthin may include beta-cryptoxanthin (3R,6′R)-4′,5′-Didehydro-5′,6′-dihydro-β,β-caroten-3-ol. The composition may aid in melanin inhibition. The composition may inhibits tyrosinase. The composition may aid in removing and preventing dandruff. The composition may be an anti-fungal agent against Malassezia furfur and Candida albicans. The composition may be an anti-yeast agent against Malassezia yeast. The composition may aid in hair growth. The composition may induce gene expression in human keratinocyte (HaCaT). The composition may aid in skin moisturization, restoring skin function by increasing the biosynthesis of hyaluronic acid. The composition may increase levels of AQP-3 mRNAs. The composition may aid in protecting skin from ultraviolet radiation, UVA radiation, and UVB radiation. The composition may provide a sun protection factor (SPF) of at least 20. The composition may prevent and treat dry eye syndrome when administered orally. The composition may increase tear break up time and reduce tear osmolarity when administered orally. The composition may increase tear production volume when administered orally. The composition may decrease defects and abrasions in the corneal epithelium when administered orally. The composition may reduce damage to ocular surfaces by reducing oxidative stress when administered orally. The composition may reduce degradation and apoptotic cell death in the cornea by reducing scrum MMP-2& MMP-9 levels when administered orally. The composition may restore the mucin layer of the eye when administered orally. The composition may reduce the expression of the IL-2, IL-6, and MMP-9 genes. The composition may down regulate the expression of the TNF-α and IL-4 genes. Extracting the carotenoids may include extracting the carotenoids at a temperature ranging from 40° C. to 90° C. for a time period ranging from 4 hours to 8 hours, enriching the carotenoids may include enriching the carotenoids using super critical fluid extraction at a temperature ranging from 40° C. to 60° C., enriching the carotenoids may include enriching the carotenoids using super critical fluid extraction at a pressure ranging from 25 mPa to 50 mPa, and purifying the carotenoids may include purifying the carotenoids using a solvent for counter current extraction comprising ethyl acetate or isopropyl acetate. Purifying the enriched carotenoids may include removing an organic layer comprising at least 50% capsanthin and adjusting a pH of a remaining aqueous layer via an acid, and purifying the carotenoids using a solvent may include purifying the carotenoids using a solvent for counter current extraction comprising ethyl acetate or isopropyl acetate such that the extraction includes capsanthin in the range from 1% to 1.5%, zeaxanthin in the range from 0.5% to 15%, and cryptoxanthin in the range from 0.1% to 0.5%. The composition may aid in removing and preventing dandruff and may be an anti-fungal agent against Malassezia furfur and Candida albicans. The composition may aid in hair growth and induces gene expression in human keratinocyte (HaCaT). The composition may aid in skin moisturization and increases levels of AQP-3 mRNAs. The composition may prevent and treat dry eye syndrome when administered orally. The composition may increase tear break up time and reduces tear osmolarity when administered orally. The composition may increase tear production volume when administered orally. The composition may decreases defects and abrasions in the corneal epithelium when administered orally. The composition may reduce damage to ocular surfaces by reducing oxidative stress when administered orally. The composition may reduce degradation and apoptotic cell death in the cornea by reducing scrum MMP-2& MMP-9 levels when administered orally. The composition may restore the mucin layer of the eye when administered orally. The composition may reduce the expression of the IL-2, IL-6, and MMP-9 genes. The composition may down regulate the expression of the TNF-α and IL-4 genes.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments in this disclosure have been described in terms of preferred embodiments, those skilled in the art will recognize that the embodiments herein can be practiced with modification within the spirit and scope of the embodiments as described herein. Numerous other modifications, equivalents, and alternatives, will become apparent to those skilled in the art once the above disclosure is fully appreciated. It is intended that the following claims be interpreted to embrace all such modifications, equivalents, and alternatives where applicable.

1. A capsicum annum extract composition comprising:
capsanthin in the range from 1%-1.5%
zeaxanthin in the range from 0.5% to 1%; and
cryptoxanthin in the range from 0.1% to 0.5%
wherein the composition is created, at least in part, from material spent from purifying carotenoids using counter current extractions; and
wherein the composition enhances skin, eyes, or hair.

2. The composition of claim 1, wherein the capsanthin comprises trans-capsanthin (3R,3′S,5′R)-3,3′-Dihydroxy-β,κ-caroten-6′-one), the zeaxanthin comprises trans-zeaxanthin (3R, 3′R-β,β-carotene-3,3′-diol), and the cryptoxanthin comprises beta-cryptoxanthin (3R,6′R)-4′,5′-Didehydro-5′,6′-dihydro-β,β-caroten-3-ol.

3. The composition of claim 1, wherein the composition aids in melanin inhibition by inhibiting tyrosinase.

4. The composition of claim 1, wherein the composition aids in removing and preventing dandruff by being an anti-fungal agent against Malassezia furfur and Candida albicans and an anti-yeast agent against Malassezia yeast.

5. The composition of claim 1, wherein the composition aids in hair growth by inducing gene expression in human keratinocyte (HaCaT).

6. The composition of claim 1, wherein the composition increases levels of AQP-3 mRNAs.

7. The composition of claim 1, wherein the composition increases tear break up time, increases tear production volume, and reduces tear osmolarity when administered orally.

8. The composition of claim 1, wherein the composition decreases defects and abrasions in the corneal epithelium and reduces damage to ocular surfaces by reducing oxidative stress when administered orally.

9. The composition of claim 1, wherein the composition reduces degradation and apoptotic cell death in the cornea by reducing serum MMP-2 & MMP-9 levels when administered orally.

10. The composition of claim 1, wherein the composition reduces the expression of the IL-2, IL-6, and MMP-9 genes and down regulates the expression of the TNF-α and IL-4 genes.

11. A method for manufacturing a composition comprising:
extracting carotenoids from spent material, the spent material created from purifying carotenoids from capsicum annum fruits using counter current extractions using a solvent or solvents;
enriching the extracted carotenoids using super critical fluid extraction; and
purifying the enriched carotenoids using counter current extractions such that the composition enhances skin, eyes, or hair.

12. The method of claim 11, wherein extracting the carotenoids comprises extracting the carotenoids at a temperature ranging from 40° C. to 90° C. for a time period ranging from 4 hours to 8 hours, wherein enriching the carotenoids comprises enriching the carotenoids using super critical fluid extraction at a temperature ranging from 40° C. to 60° C., wherein enriching the carotenoids comprises enriching the carotenoids using super critical fluid extraction at a pressure ranging from 25 mPa to 50 mPa, and wherein purifying the carotenoids comprises purifying the carotenoids using a solvent for counter current extraction comprising ethyl acetate or isopropyl acetate.

13. The method of claim 11, wherein purifying the enriched carotenoids comprises removing an organic layer comprising at least 50% capsanthin and adjusting a pH of a remaining aqueous layer via an acid, and wherein purifying the carotenoids using a solvent comprises purifying the carotenoids using a solvent for counter current extraction comprising ethyl acetate or isopropyl acetate such that the extraction comprises capsanthin in the range from 1% to 1.5%, zeaxanthin in the range from 0.5% to 15%, and cryptoxanthin in the range from 0.1% to 0.5%.

14. The method of claim 11, wherein the composition increases tear break up time and reduces tear osmolarity when administered orally.

15. The method of claim 11, wherein the composition increases tear production volume when administered orally.

16. The method of claim 11, wherein the composition decreases defects and abrasions in the corneal epithelium when administered orally.

17. The method of claim 11, wherein the composition reduces damage to ocular surfaces by reducing oxidative stress when administered orally.

18. The method of claim 11, wherein the composition reduces degradation and apoptotic cell death in the cornea by reducing serum MMP-2 & MMP-9 levels when administered orally.

19. The method of claim 11, wherein the composition reduces the expression of the IL-2, IL-6, and MMP-9 genes and down regulates the expression of the TNF-α and IL-4 genes.

20. A method for manufacturing a composition comprising:
extracting carotenoids from spent material, the spent material created from purifying carotenoids from capsicum annum fruits using counter current extractions using a solvent or solvents;
enriching the extracted carotenoids using super critical fluid extraction; and
purifying the hydrolyzed carotenoids using counter current extractions such that the composition includes capsanthin in the range from 1% to 1.5%, zeaxanthin in the range from 0.5% to 15%, and cryptoxanthin in the range from 0.1% to 0.5% such that the composition enhances skin, eyes, or hair.
